(19) United States
(12) Patent Application Publication
(10) Pub. No.: US 2019/0239842 A1
(43) Pub. Date: Aug. 8, 2019

(54) SYSTEM FOR PROVIDING IMAGES FOR ENDOLEAK DETECTION

(71) Applicant: KONINKLIJKE PHILIPS N.V., EINDHOVEN (NL)

(72) Inventors: Roland PROKSA, NEU WULMSTORF (DE); Peter Benjamin NOEL, UNTERFÖHRING (DE); Daniela MUENZEL, MÜNCHEN (DE); Julia DANGELMAIER, MÜNCHEN (DE)

(21) Appl. No.: 16/314,742

(22) PCT Filed: Jun. 29, 2017
(86) PCT No.: PCT/EP2017/066083

§ 371 Date: Jan. 2, 2019

(30) Foreign Application Priority Data
Jul. 6, 2016 (EP) 16178251.1

(51) Int. Cl. A61B 6/00; A61B 6/03; A61F 2/07

(52) U.S. Cl. CPC A61B 6/507; A61B 6/037; A61B 6/5235; A61B 6/5217; A61F 2/07

(57) ABSTRACT

The invention relates to a system for providing images for detecting an endoleak after a stent graft has been placed within the aorta of a person at a stent graft location. First and second contrast agents are injected at first and second times (T1, T2) and first, second and third computed tomography images of the stent graft location showing a) the first contrast agent, b) the second contrast agent and c) the stent graft and tissue, respectively, are reconstructed based on spectral projection data acquired at a third time (T3). By using these computed tomography images for detecting an endoleak, the endoleak detection can be significantly improved, wherein this improved endoleak detection can be provided with a relative low radiation dose, because the images can be reconstructed by using only the computed tomography scan at the third time.

### FIELD OF THE INVENTION

The invention relates to a system, method and computer program for providing images for detecting an endoleak after a stent graft has been placed within the aorta of a person.

### BACKGROUND OF THE INVENTION

US 2004/0101088 A1 discloses a multi-energy computed tomography system comprising a radiation source, a radiation detector and a computer coupled to the radiation source and the radiation detector, wherein the computer is configured to acquire a plurality of projection data of an area of interest in flow communication with a first vessel and a second vessel. The computer is further configured to decompose the projection data into a first density map representative of a first contrast agent introduced into the first vessel and a second density map representative of a second contrast agent introduced into the second vessel.

The article “Dual-energy computed tomography after endovascular aortic aneurysm repair: The role of hard plaque imaging for endoleak detection” by R. Miiller-Wille et al., European Radiology, Springer International, volume 24, pages 2449 to 2457 (2014) discloses a dual-energy computed tomography imaging technique for detecting endoleaks after an endovascular aneurysm repair (EVAR) procedure.

The article “Endoleak Detection After Endovascular Repair of Thoracic Aortic Aneurysm Using Dual-Source Dual-Energy CT: Suitable Scanning Protocols and Potential Radiation Dose Reduction” by L. Flors et al., American Journal of Roentgenology, volume 200, pages 451 to 460 (2013) discloses a dual-source dual-energy computed tomography (DECT) system which is used for detecting endoleaks after a thoracic endovascular aortic repair procedure has been performed. This procedure includes placing a stent graft within the aorta of a person. The DECT system generates a first image showing a distribution of a contrast agent at a stent graft location, i.e. at the location within the person at which the stent graft has been placed, and a second image of the stent graft location, which shows the stent graft and the surrounding of the stent graft without the contrast agent. These two images are used for detecting endoleaks.

### SUMMARY OF THE INVENTION

It is an object of the present invention to provide a system, method and computer program for providing images for detecting an endoleak, after a stent graft has been placed within the aorta of a person, which allows for an improved detection of the endoleak.

In a first aspect of the present invention a system for providing images for detecting an endoleak, after a stent graft has been placed within the aorta of a person at a stent graft location, is presented, wherein the system comprises:
a spectral computed tomography projection data acquisition unit for acquiring, after a first contrast agent has been injected at a first time and a second contrast agent has been injected at a second time into the person, spectral computed tomography projection data showing the stent graft location at a third time, wherein the spectral computed tomography projection data correspond to at least three different energy spectra, and
a reconstruction unit for reconstructing, based on the acquired spectral computed tomography projection data, a first computed tomography image showing the first contrast agent at the stent graft location, a second computed tomography image showing the second contrast agent at the stent graft location and a third computed tomography image showing the stent graft and tissue of the person at the stent graft location without the first and second contrast agents.

By using these computed tomography images for detecting an endoleak, especially the first and second computed tomography images representing different flow times of the contrast agents, the endoleak detection can be significantly improved, wherein this improved endoleak detection can be provided with a relative low radiation dose, because the images can be reconstructed by using only the computed tomography scan at the third time.

The first, second and third times are preferentially start times at which the respective procedure starts. In particular, the first time is preferentially the time at which the injection of the first contrast agent starts, the second time is preferentially the time at which the injection of the second contrast agent starts and the third time is preferentially the time at which the acquisition of the spectral computed tomography projection data starts. However, the first, second and third times can also be defined in another way, wherein preferentially the first and second times are defined in the same way, i.e., for instance, if the first time is defined as being the start time of the injection of the first contrast agent, the second time is preferentially defined as being the start time of the injection of the second contrast agent. At the third time the first contrast agent and the second contrast agent are at the stent graft location, i.e. at the third time the first and second contrast agents have flowed to the location at which the spectral computed tomography projection data are acquired, i.e. to the stent graft location.

The spectral computed tomography projection data acquisition unit comprises a radiation unit emitting radiation traversing the person and a detector for detecting the radiation after having traversed the person and for generating the spectral computed tomography projection data based on the detected radiation. The radiation unit and preferentially also the detector are rotatable around the person, in order to allow for the acquisition of the spectral computed tomography projection data in different directions. The spectral computed tomography projection data correspond to at least three different energy spectra of the radiation emitted by the radiation source, i.e. for each energy spectrum a set of computed tomography projection data is provided. The detector can be a spectral detector like a photon counting detector and the radiation unit can be adapted to emit polychromatic radiation, wherein the spectral detector is adapted to provide at least three different sets of computed tomography projection data which correspond to at least three different energy spectra, i.e., for instance, to at least three different energy bins. The radiation unit and the detector can also be adapted in another way for providing the spectral computed tomography projection data. For example, a non-spectral detector and a radiation unit subsequently emitting radiation having different energy spectra can be used, wherein the radiation unit is able to provide radiation with at least three different energy spectra. The radiation unit can comprise, for instance, three or more x-ray tubes subsequently operable at different voltages.

Preferentially, the system further comprises a contrast agent tracking unit for tracking the first contrast agent within the person at a detection location being at or proximal to the stent graft location and a time determining unit for determining the second time and/or the third time based on the tracking of the first contrast agent. The time determining unit can be further adapted to provide the second time to an injector for injecting the second contrast agent at the determined second time and/or to provide the third time to the spectral computed tomography projection data acquisition unit for acquiring the spectral computed tomography projection data at the determined third time. In particular, the time determining unit can be adapted to determine a peak time at which the first contrast agent reaches its peak at the detection location and to determine the second time and/or the third time based on the determined peak time. This allows for an adaptation of the injection of the second contrast agent and/or of the acquisition of the spectral computed tomography projection data based on the actual flow properties of the injected first contrast agent within the person. This adaptation of the second time and/or the third time can lead to first and/or second computed tomography images showing more of the first and/or second contrast agents, respectively, which in turn can lead to a further improved detection of the endo leak. Preferentially, the detection location is directly proximal, i.e. proximally adjacent, to the stent graft location.

It should be understood that a determination of the second time and/or the third time means that a) the second time, b) the third time or c) the second time and the third time are determined, i.e. that the expression “A and/or B” refers to “A, B, or A and B”. Also the expression “at least one of A and B” could be used instead, i.e. also this expression is to be understood as meaning “A, B, or A and B”.

In an embodiment the time determining unit can be adapted to determine the second time such that it corresponds to a difference of a) the third time and b) a difference between the peak time and the first time. The third time can be predefined by predefining a delay time period between the first time and the third time. The delay time period may be predefined by a user, i.e. the user may input a desired delay time period or modify an already given delay time period, wherein the already given delay time period may have been stored in the system, especially in the time determining unit. The system, particularly the time determining unit, may provide a user interface for allowing the user to define the delay time period. Preferentially, the delay time period is predefined such that an imaging of the first contrast agent in the aneurysm sac is optimized, wherein, if the second time is calculated such that it corresponds to a difference of a) the third time and b) a difference between the peak time and the first time, also the imaging of the second contrast agent can be optimized.

The contrast agent tracking unit can be adapted to cooperate with the spectral computed tomography projection data acquisition unit such that a) the spectral computed tomography projection data acquisition unit acquires projection data at different times, which might be predefined, wherein the acquired projection data show the detection location, and b) the contrast agent tracking unit detects the amount of the first contrast agent in the acquired projection data, in order to track the first contrast agent within the person at the detection location over time. In particular, first projection data can be acquired, before the first contrast agent has reached the detection location, and these first projection data can be subtracted from projection data which have been acquired at different times, after at least a part of the first contrast agent has reached the detection location, in order to calculate corresponding subtraction projection data for the different times, wherein the projection values of these subtraction projection data are indicative for the amount of the first contrast agent within the person at the detection location. For instance, the contrast agent tracking unit can be adapted to calculate the sum of the projection data values of subtraction projection data determined for a respective time or calculate another value based on these projection data values, in order to track the first contrast agent within the person at the detection location. The projection data, which are used for tracking the first contrast agent, can be spectral or non-spectral projection data. Moreover, they can be acquired at a single position of the radiation unit of the spectral computed tomography projection data acquisition unit.

The contrast agent tracking unit can also cooperate with the reconstruction unit such that a) the reconstruction unit reconstructs computed tomography images based on the projection data acquired at different times, in order to provide computed tomography images for different times, and b) the contrast agent tracking unit detects the amount of the first contrast agent in the reconstructed computed tomography images, in order to track the first contrast agent within the person at the detection location over time. Also in this case the projection data, which are used for tracking the first contrast agent, can be spectral or non-spectral projection data. If they are spectral, the reconstruction unit can reconstruct computed tomography images for the different times, which only show the first contrast agent at the detection location, wherein these computed tomography images can be used for tracking the first contrast agent at the detection location over time.

The injector can be regarded as being a component of the system for providing images for detecting an endo leak or it can be regarded as being a separate component which is not part of this system. The injector is preferentially adapted to inject the first contrast agent and the second contrast agent at a same injection location on the person.

The system can comprise an endo leak detection unit for detecting the endoleak based on the first computed tomography image and/or the second computed tomography image and/or the third computed tomography image. The endoleak detection unit can be adapted to allow for a fully automatic, semi-automatic or manual detection of the endoleak based on at least one of the computed tomography images. In the latter case the endoleak detection unit may be adapted to show the first, second and third computed tomography images on a display and it may provide a graphical user interface, in order to allow a user like a physician to indicate an endo leak in one or more of these computed tomography images.

In an embodiment the endo leak detection unit is adapted to automatically detect the endoleak based on a) the first computed tomography image and/or the second computed tomography image and b) the third computed image. In particular, the endoleak detection unit can be adapted to i) detect the stent graft in the third computed tomography image at the stent graft location, ii) determine whether the first contrast agent and/or the second contrast agent is outside of the stent graft at the stent graft location in the first computed tomography image and/or in the second computed tomography image by using the detection of the stent graft in the third computed tomography image, and iii) detect the endoleak, if it has been determined that the first contrast agent and/or the second contrast agent is outside of the stent graft.

The endoleak detection unit is preferentially further adapted to classify the detected endoleak into at least one of a predefined group of endoleak classes based on the first computed tomography image and/or the second computed tomography image and/or the third computed tomography image. In particular, the endoleak detection unit is adapted to classify the detected endoleak into at least one of the predefined group of endoleak classes based on the location of the first contrast agent in the first computed tomography image and/or the location of the second contrast agent in the second computed tomography image relative to the stent graft detected in the third computed tomography image. For instance, the endoleak detection unit can comprise classification rules defining assignments between endoleak classes and locations of the respective contrast agent relative to the stent graft and optionally also relative to the vessel, in which the stent graft is placed, wherein the endoleak detection unit can be adapted to determine the respective endoleak class based on the location of the respective contrast agent and the classification rules. This allows for a very reliable and accurate classification of the endoleak.

In another aspect of the present invention a method for providing images for detecting an endoleak, after a stent graft has been placed within the aorta of a person at a stent graft location, is presented, wherein the method comprises:
acquiring, after a first contrast agent has been injected at a first time and a second contrast agent has been injected at a second time into the person, spectral computed tomography projection data showing the stent graft location at a third time by a spectral computed tomography projection data acquisition unit, wherein the spectral computed tomography projection data correspond to at least three different energy spectra, and
reconstructing, based on the acquired spectral computed tomography projection data, a first computed tomography image showing the first contrast agent at the stent graft location, a second computed tomography image showing the second contrast agent at the stent graft location, and a third computed tomography image showing the stent graft and tissue of the person at the stent graft location without the first and second contrast agents by a reconstruction unit.

In a further aspect of the present invention a computer program for providing images for detecting an endoleak, after a stent graft has been placed within the aorta of a person at a stent graft location, is presented, wherein the computer program comprises program code means for causing a system as defined in claim 1 to carry out the method as defined in claim 10, when the computer program is run on the system.

It shall be understood that the system of claim 1, the method of claim 10 and the computer program of claim 11 have similar and/or identical preferred embodiments, in particular, as defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily an embodiment of a system for detecting an endoleak, after a stent graft has been placed within the aorta of a person at a stent graft location,

FIG. 2 shows schematically and exemplarily the stent graft within the aorta,

FIG. 3 illustrates schematically and exemplarily a first time at which a first contrast agent is injected, a second time at which a second contrast agent is injected and a third time at which spectral computed tomography projection data are acquired, and

FIG. 4 shows a flowchart exemplarily illustrating an embodiment of a method for detecting an endoleak, after a stent graft has been placed within the aorta of a person at a stent graft location.

### DETAILED DESCRIPTION OF EMBODIMENTS

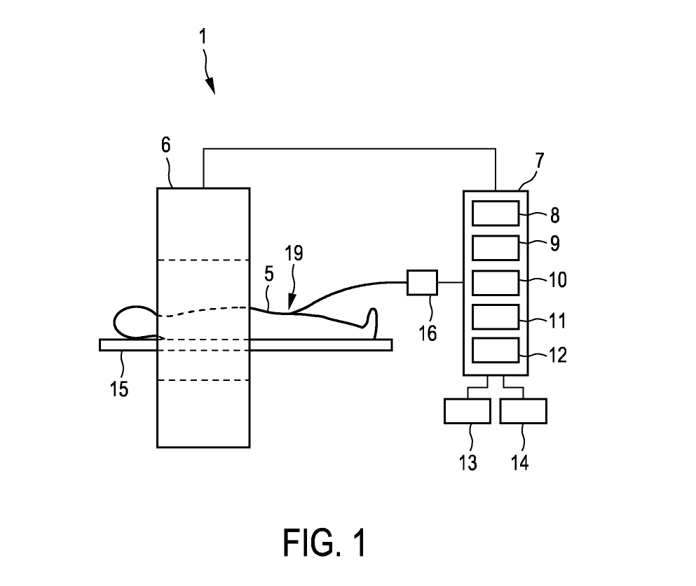

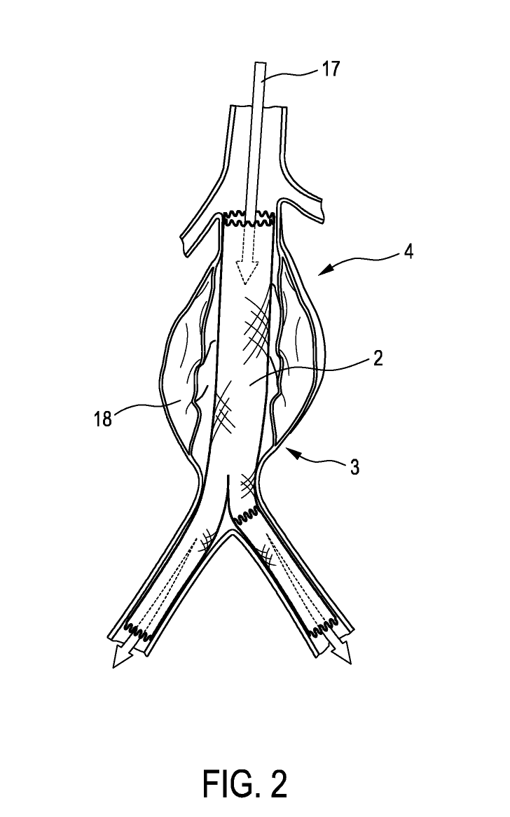

FIG. 1 shows schematically and exemplarily an embodiment of a system 1 for providing images for detecting an endoleak, after an endovascular stent graft 2 has been placed within the aorta 3 of a person 5 at a stent graft location 4 during an EVAR procedure as schematically and exemplarily illustrated in FIG. 2. If the EVAR procedure has been performed correctly and if there are no complications, the blood flows through the stent graft 2 as indicated in FIG. 2 by the arrow 17. However, if there is a leak into an aneurysm sack 18, i.e. if there is an endoleak, the aneurysm sack 18 may be further perfused and pressurized, thereby conferring an ongoing risk of aneurysm enlargement and/or rupture.

The system 1 comprises an injector 16 for injecting a first contrast agent at a first time and for injecting a second contrast agent at a second time into the person 5 at a same injection location 19. In another embodiment the injector 16 may also be adapted to inject the first contrast agent and the second contrast agent at different injection locations. For instance, the injector 16 can comprise a first sub injector for injecting the first contrast agent at a first injection location and a second sub injector for injecting the second contrast agent at a second injection location.

The system 1 further comprises a spectral computed tomography projection data acquisition unit 6 for acquiring, after the first contrast agent has been injected at the first time and the second contrast agent has been injected at the second time into the person at the injection location 19, spectral computed tomography projection data showing the stent graft location 4 at a third time, wherein the spectral computed tomography projection data correspond to at least three different energy spectra. Thus, the spectral computed tomography projection data acquisition unit acquires at least three different sets of spectral computed tomography projection data which correspond to at least three different energy spectra, i.e., for instance, to at least three different energies or energy bins of a photon counting detector with energy discrimination.

The spectral computed tomography projection data acquisition unit 6 comprises a radiation unit and a detector which are arranged on opposing sides of a rotatable gantry, wherein the gantry is rotated around the person 5, who lies on a support means 15 like a patient table, for acquiring the spectral computed tomography projection data in different acquisition directions. The radiation emitted by the radiation unit is preferentially a cone beam and the detector preferentially has a two-dimensional detection surface such that the spectral computed tomography projection data are cone beam data. However, the radiation unit can also be adapted to emit a fan beam and the detector can have a one-dimensional arrangement of detection elements such that the spectral computed tomography projection data can be fan beam data.

In order to acquire the spectral computed tomography projection data such that they correspond to at least three different energy spectra, the radiation unit can comprise three x-ray tubes emitting x-rays having different energy spectra. These x-ray tubes can be operated such that only a single one of these x-ray tubes emits radiation at a time such that it is clear that the detected radiation corresponds to the energy spectrum of the radiation emitted by the respective x-ray tube. In this way computed tomography projection data corresponding to different energy spectra can be distinguished from each other. The spectral computed tomography projection data acquisition unit can also be adapted to distinguish computed tomography projection data, which correspond to different energy spectra, in another way. For instance, the radiation unit can be adapted to emit polychromatic radiation, wherein the detector can be a spectral detector for distinguishing at least three different energy spectra of the emitted radiation. For instance, the spectral detector can be a multi-layer detector, wherein different detection layers are stacked on top of each other and wherein in each layer x-rays having a certain energy spectrum are detected. In this example the spectral detector would have at least three different detection layers.

The spectral computed tomography projection data acquisition unit 6 is connected to a processing and control device 7 of the system 1. The processing and control device 7 includes a reconstruction unit 10 for reconstructing, based on the acquired spectral computed tomography projection data, a first computed tomography image showing only the first contrast agent at the stent graft location, a second computed tomography image showing only the second contrast agent at the stent graft location 4 and a third computed tomography image showing the stent graft 2 and tissue of the person 5 at the stent graft location 4 without the first and second contrast agents. The reconstruction unit 10 uses known reconstruction algorithms like a filtered back protection algorithm and a material decomposition technique, in order to reconstruct the different computed tomography images. Known reconstruction techniques, which might be used by the reconstruction unit 10, are disclosed, for instance, in the article “K-edge imaging in x-ray computed tomography using multi-bin photon counting detectors” by E. Roessl and R. Proksa, Physics in Medicine and Biology, volume 52, pages 4679 to 4696 (2007), which is herewith incorporated by reference.

The system 1 further comprises an endo leak detection unit 11 for detecting the endo leak based on the first computed tomography image and/or the second computed tomography image and/or the third computed tomography image. Moreover, the system 1 comprises a contrast agent tracking unit 8 for tracking the first contrast agent within the person 5 at a detection location being at or directly proximal to the stent graft location 4 and a time determining unit 9 for a) determining the second time and/or the third time based on the tracking of the first contrast agent and b) providing the second time to the injector 16 for injecting the second contrast agent at the determined second time and/or to the spectral computed tomography projection data acquisition unit 6 for acquiring the spectral computed tomography projection data at the determined third time. In this embodiment the time determining unit 9 is adapted to determine a peak time at which the first contrast agent reaches its peak at the detection location and to determine the second time and/or the third time based on the determined peak time.

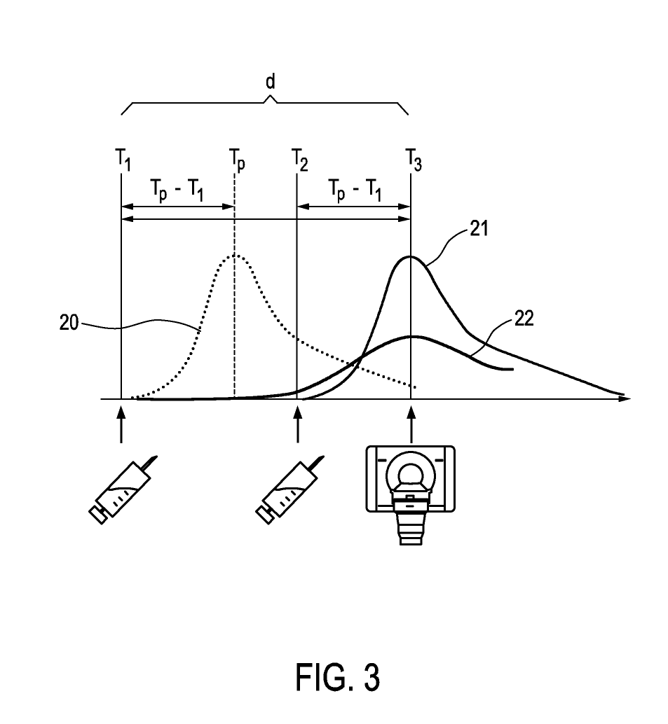

The time determining unit 9 preferentially provides a user interface allowing a user to input a desired delay time period between the first time and the third time. This delay time period may be chosen such that imaging of the first contrast agent in the aneurysm sac is optimized. The time determining unit 9 is preferentially further adapted to determine the second time such that it corresponds to the difference of a) the third time defined by the first time and the input delay time period and b) the difference between the peak time and the first time, in order to also optimize the imaging of the second contrast agent. In the following this will be explained in more detail with reference to FIG. 3.

At the first time T₁ the first contrast agent is injected at the injection location 19. The contrast agent tracking unit 8 tracks the injected first contrast agent at the detection location, wherein in FIG. 3 the curve 20 denotes the corresponding amount of contrast agent within the stent graft 2, i.e. the curve 20 denotes the intraluminal, with respect to the stent graft, amount of the first contrast agent. The time determining unit 9 determines the peak time of the curve 20, i.e. the time Tₚ at which the curve 20 reaches its peak.

The third time T₃, at which the spectral computed tomography projection data are acquired, is defined by the sum of the first time T₁ and the delay time period d input by the user. The delay time period is preferentially 350 s or smaller, further preferred 300 s or smaller, further preferred 250 s or smaller, and even further preferred 200 s or smaller. In a preferred embodiment the delay time period d is within the range of 60 to 120 s. The second time T₂, at which the second contrast agent is injected at the injection location 19, is defined by the difference between a) the third time T₃ and b) the difference between the peak time Tₚ and the first time T₁. The curve 21 denotes the amount of the second contrast agent at the stent graft location 4 over time. Also the curve 21 refers to the intraluminal, with respect to the stent graft 2, amount of the second contrast agent, i.e. the amount of the second contrast agent within the stent graft 2. As can be seen in FIG. 3, by determining the second time T₂ as described above, it can be ensured that the amount of the second contrast agent within the stent graft 2 reaches its maximum at the third time T₃. Moreover, since the delay time period d has been chosen accordingly, also the amount of the first contrast agent within the aneurysm sack reaches its maximum at the third time T₃ as denoted by the curve 22 in FIG. 3, i.e. the curve 22 shows the extraluminal, with respect to the stent graft 2, amount of the first contrast agent.

In this embodiment the endo leak detection unit 11 is adapted to automatically detect an endo leak based on a) the first computed tomography image and/or the second computed tomography image and b) the third computed tomography image. In particular, the endo leak detection unit 11 is adapted to a) detect the stent graft 2 in the third computed tomography image at the stent graft location 4, b) determine whether the first contrast agent and/or the second contrast agent is outside of the stent graft 2 at the stent graft location 4 in the first computed tomography image and/or in the second computed tomography image by using the detection of the stent graft 2 in the third computed tomography image, and c) detect the endo leak if it has been determined that the first contrast agent and/or the second contrast agent is outside of the stent graft 2. If an endo leak has been detected, this can be shown on a display 14. For instance, the display 14 can show the first, second and third computed tomography images and provide an indication on these images, which marks the location of the endoleak.

For tracking the first contrast agent within the person 5 at the detection location the spectral computed tomography projection data acquisition unit 6 can be adapted to acquire projection data at a fixed rotational position of the radiation unit and the detector and the contrast agent tracking unit 8 can be adapted to track the first contrast agent within the person 5 at the detection location based on these projection data. The projection data can be spectral or non-spectral and they may correspond to a one-dimensional or a two-dimensional distribution of detection elements. If they correspond to a one-dimensional distribution of detection elements and if the detector has a two-dimensional distribution of detection elements, the radiation unit may collimate the emitted radiation such that only a single line of the detection elements of the two-dimensional distribution of detection elements is illuminated. These projection data are acquired at different times, in order to allow the contrast agent tracking unit 8 to track the first contrast agent within the person 5 at the detection location over time. The spectral computed tomography projection data acquisition unit 6 may provide the projection data already, before the first contrast agent has reached the detection location, such that it provides projection data with the first contrast agent and projection data without the first contrast agent, wherein the contrast agent tracking unit 8 can calculate subtraction projection data for different times by subtracting projection data, which show the first contrast agent and which have been acquired at different times, from projection data which have been acquired, before the first contrast agent has reached the detection location. Based on these subtraction projection data the contrast agent tracking unit 8 may track the first contrast agent. For instance, the contrast agent tracking unit 8 can be adapted to calculate the sum of the projection values of the subtraction projection data, in order to provide a time-dependent sum function, wherein this time-dependent sum function can be used for indicating the amount of the first contrast agent over time.

The endo leak detection unit 11 is further adapted to classify the detected endo leak into at least one of a predefined group of endo leak classes based on the first computed tomography image and/or the second computed tomography image and/or the third computed tomography image. In particular, the endoleak detection unit 11 is adapted to classify the detected endoleak into at least one of the predefined group of endoleak classes based on the location of the first contrast agent in the first computed tomography image and/or the location of the second contrast agent in the second computed tomography image relative to the stent graft 2 detected in the third computed tomography image. Preferentially, the endo leak detection unit 11 comprises classification rules comprising assignments between a) locations of the first contrast agent and/or the second contrast agent relative to the stent graft as determinable based on the first, second and third computed tomography images and b) the endo leak classes, wherein the endoleak detection unit 11 is adapted to determine the locations of the first contrast agent and/or the second contrast agent relative to the stent graft based on the computed tomography images and to classify a detected endoleak based on these determined locations and the classification rules.

Preferentially, five endoleak classes are predefined, i.e. a first class referring to attachment site leaks, a second class referring to collateral vessel leaks, a third class referring to a graft failure, a fourth class referring to a graft wall porosity, and a fifth class referring to endotension. The first class includes a perigraft leakage at proximal or distal graft attachment sites. The second class includes a retrograde flow to the aneurysm sack from branches such as the lumbar and inferior mesenteric arteries. The third class includes a leakage between overlapping parts of the stent graft or a rupture through graft material. The fourth class includes a leakage through the graft wall due to the quality, i.e. the porosity, of the graft material, and the fifth class refers to an expansion of the aneurysm sack without an identifiable leak.

The system 1 further comprises a controller 12 for controlling the several components, especially the spectral computed tomography projection data acquisition unit 6, the injector 16 and the components of the processing and control device 7. Moreover, the system 1 further comprises an input unit 13 like a keyboard, a computer mouse, a touchpad, et cetera, in order to allow a user to make inputs into the system 1.

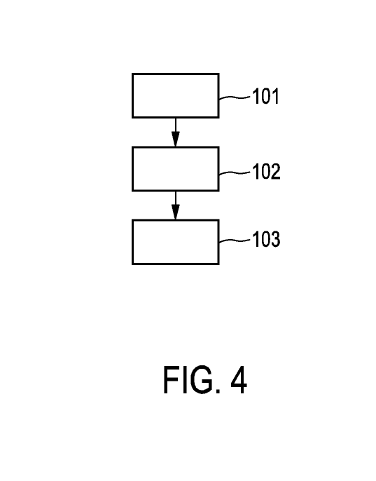

In the following an embodiment of a method for providing images for detecting an endoleak, after a stent graft has been placed within the aorta of a person at a stent graft location, will exemplarily be described with reference to a flowchart shown in FIG. 4.

After a first contrast agent has been injected at a first time and a second contrast agent has been injected at a second time into the person, in step 101 spectral computed tomography projection data showing the stent graft location are acquired at a third time by the spectral computed tomography projection data acquisition unit 6, wherein the spectral computed tomography projection data correspond to at least three different energy spectra. In step 102 the reconstruction unit 10 reconstructs, based on the acquired spectral computed tomography projection data, a first computed tomography image showing only the first contrast agent at the stent graft location, a second computed tomography image showing only the second contrast agent at the stent graft location and a third computed tomography image showing the stent graft and tissue at the stent graft location without the first and second contrast agents. In step 103 the endoleak is detected based on the first computed tomography image and/or the second computed tomography image and/or the third computed tomography image by the endoleak detection unit 11.

The first and second contrast agents preferentially have different K-edges, wherein one of these contrast agents may be gadolinium based and the other of these contrast agents may be iodine based. The K-edges preferentially are within the energy spectra to which the spectral computed tomography projection data correspond. The acquired spectral computed tomography projection data undergo a material decomposition and reconstruction yielding a set of computed tomography images containing maps of the first contrast agent and the second contrast agent, i.e. the first and the second computed tomography images, and a virtual-non-contrast (VMC) map, i.e. the third computed tomography image. The material delineation leads to the effect that for generating the different maps, i.e. the different computed tomography images, a single computed tomography scan is sufficient. The first computed tomography image can be regarded as representing late image information, the second computed tomography image can be regarded as representing arterial image information and the third computed tomography image can be regarded as showing no contrast image information. By using only a single computed tomography scan for providing these different kinds of information this information can be provided with a relatively low x-ray dose. Moreover, the three computed tomography images perfectly match in space. By using the tracking of the first contrast agent, in particular, by performing a bolus tracking procedure with respect to the first contrast agent, the imaging scan can be optimally tuned to the expected maximal contrast uptake of the second contrast agent. This can increase the contrast, makes the acquisition more robust and may therefore require less contrast agent. In this case the scan time parameters, especially the above mentioned second time T₂ and third time T₃, can be automatically determined.

EVAR is a type of endovascular surgery used to treat pathology of the aorta, most commonly an abdominal aortic aneurysm. The procedure involves the placement of a stent graft, which might be expandable, within the aorta, in order to treat aortic disease without operating directly on the aorta. After the stent graft has been placed within the aorta, one or several endo leaks might be present.

Endoleaks generally have variable flow rates such that they can be detected at variable times after contrast agent injection. For this reason it is also possible to use a multiphasic computed tomography angiogram, wherein a corresponding protocol may include imaging before the administration of a contrast agent, imaging after the administration of the contrast agent in the arterial phase, and imaging in a post-contrast delayed phase. Pre-contrast images can be helpful in differentiating classification in the aneurysm sack from an endoleak, thereby reducing the number of indeterminate studies. Delayed phase imaging can demonstrate endoleaks that are not visualized during the arterial phase. However, endoleaks can also be detected in arterial phase images and they can be important in planning exactly where to access the endo leak during, for instance, a trans-lumbar embolization.

The system and method for providing images for detecting an endoleak described above with reference to FIGS. 1 to 4 provide a new imaging technique for endoleak detection and classification. The technique uses a spectral computed tomography scanner which provides a sufficient spectral separation for K-edge imaging, wherein the detector might be a spectral detector like a photon counting detector providing three or more energy bins. Such a scanner provides the ability for selective imaging of at least two different contrast agents with a detectable K-edge contrast payload.

Although in above described embodiments the system and method are adapted to generate three computed tomography images corresponding to a) a first contrast agent, b) a second contrast agent and c) the stent graft and the tissue at the stent graft location without any contrast agent, in other embodiments more than two contrast agents can be used and correspondingly more than three computed tomography images can be reconstructed. If more than two contrast agents are used, for each contrast agent a corresponding computed tomography image can be reconstructed and used for detecting and optionally also classifying the endo leak.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage. Operations like the reconstruction of the computed tomography images, the detection of the endoleak, the classification of the detected endoleak, the tracking of the first contrast agent, the determining of the second time and/or the third time, et cetera performed by one or several units or devices can be performed by any other number of units or devices. These operations and/or the control of the system for providing images for detecting an endo leak in accordance with the method for providing images for detecting an endo leak can be implemented as program code means of computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

The invention relates to a system for providing images for detecting an endo leak after a stent graft has been placed within the aorta of a person at a stent graft location. First and second contrast agents are injected at first and second times and first, second and third computed tomography images of the stent graft location showing a) the first contrast agent, b) the second contrast agent, and c) the stent graft and tissue, respectively, are reconstructed based on spectral projection data acquired at a third time. By using these computed tomography images for detecting an endoleak, the endo leak detection can be significantly improved, wherein this improved endoleak detection can be provided with a relative low radiation dose, because the images can be reconstructed by using only the computed tomography scan at the third time.

1. A system for providing images for detecting an endoleak after a stent graft has been placed within the aorta of a person at a stent graft location, the system comprising:
a spectral computed tomography projection data acquisition unit for acquiring, after a first contrast agent has been injected at a first time and a second contrast agent has been injected at a second time into the person, spectral computed tomography projection data showing the stent graft location at a third time, wherein the spectral computed tomography projection data correspond to at least three different energy spectra, and
a reconstruction unit for reconstructing, based on the acquired spectral computed tomography projection data, a first computed tomography image showing the first contrast agent at the stent graft location, a second computed tomography image showing the second contrast agent at the stent graft location and a third computed tomography image showing the stent graft and tissue of the person at the stent graft location without the first and second contrast agents.

2. The system as defined in claim 1, wherein the system further comprises:
a contrast agent tracking unit for tracking the first contrast agent within the person at a detection location being at or proximal to the stent graft location, and
a time determining unit for determining the second time and/or the third time based on the tracking of the first contrast agent.

3. The system as defined in claim 2, wherein the time determining unit is adapted to determine a peak time at which the first contrast agent reaches its peak at the detection location and to determine the second time and/or the third time based on the determined peak time.

4. The system as defined in claim 3, wherein the time determining unit is adapted to determine the second time such that it corresponds to a difference of a) the third time and b) a difference between the peak time and the first time.

5. The system as defined in claim 1, wherein the system further comprises an endoleak detection unit for detecting the endoleak based on the first computed tomography image and/or the second computed tomography image and/or the third computed tomography image.

6. The system as defined in claim 5, wherein the endoleak detection unit is adapted to automatically detect the endoleak based on a) the first computed tomography image and/or the second computed tomography image and b) the third computed image.

7. The system as defined in claim 6, wherein the endoleak detection unit is adapted to:
detect the stent graft in the third computed tomography image at the stent graft location,
determine whether the first contrast agent and/or the second contrast agent is outside of the stent graft at the stent graft location in the first computed tomography image and/or in the second computed tomography image by using the detection of the stent graft in the third computed tomography image, and
detect the endoleak, if it has been determined that the first contrast agent and/or the second contrast agent is outside of the stent graft.

8. The system as defined in claim 5, wherein the endoleak detection unit is further adapted to classify the detected endoleak into at least one of a predefined group of endoleak classes based on the first computed tomography image and/or the second computed tomography image and/or the third computed tomography image.

9. The system as defined in claim 8, wherein the endoleak detection unit is further adapted to classify the detected endoleak into at least one of the predefined group of endoleak classes based on the location of the first contrast agent in the first computed tomography image and/or the location of the second contrast agent in the second computed tomography image relative to the stent graft detected in the third computed tomography image.

10. A method for providing images for detecting an endoleak, after a stent graft has been placed within the aorta of a person at a stent graft location the method comprising:
acquiring, after a first contrast agent has been injected at a first time and a second contrast agent has been injected at a second time into the person, spectral computed tomography projection data showing the stent graft location at a third time by a spectral computed tomography projection data acquisition unit, wherein the spectral computed tomography projection data correspond to at least three different energy spectra, and
reconstructing, based on the acquired spectral computed tomography projection data, a first computed tomography image showing the first contrast agent at the stent graft location, a second computed tomography image showing the second contrast agent at the stent graft location, and a third computed tomography image showing the stent graft and tissue of the person at the stent graft location without the first and second contrast agents.

11. A computer program for providing images for detecting an endoleak, after a stent graft has been placed within the aorta of a person at a stent graft location, the computer program comprising program code means for causing a system to carry out the method as defined in claim 10, when the computer program is run on the system.
